(19) United States
(12) Patent Application Publication
(10) Pub. No.: US 2005/0165339 A1
(43) Pub. Date: Jul. 28, 2005

(54) ARTICLE FOR TREATMENT AND PREVENTION OF SHIN SPLITS

(76) Inventor: Murray R. Davidson, San Diego, CA (US)

Correspondence Address: LAW OFFICE OF MICHAEL P. EDDY, #510, 2683 VIA DE LA VALLE, SUITE G, DEL MAR, CA, 92014, US

(21) Appl. No.: 10/908,021

(22) Filed: Apr. 25, 2005

(51) Int. Cl. A61F 5/00

(52) U.S. Cl. 602/27

(57) ABSTRACT

An article for treatment and prevention of shin splints including a lower leg and ankle brace having a generally tubular body with an upper and a lower end shaped to fit about a wearer's leg extending from the bottom of the foot to below the wearer's knee, and a heel cup having side walls, a rear wall, and a bottom wall defining a recess located in the heel area, said heel cup being shaped to fit about the sides and rear of a wearer's heel, wherein said heel cup is secured to the brace at least about the peripheral edge of the heel cup to form an integral assembly therewith to secure the heel cup to the wearer's heel whereby the brace supports the wearer's lower leg and ankle and said heel cup is maintained in a controlled position on the foot in order to absorb shock.

### BACKGROUND

Embodiments of the present invention relate to shin splints, or pain and tenderness that are present on the lower leg. When shin splints are present, running and walking may be extremely painful. In severe cases, even light weight bearing may be painful.

### SUMMARY

An article for treatment and prevention of shin splints including a lower leg brace having a generally tubular body with an upper and a lower end shaped to fit about a wearer's leg extending from the bottom of the foot to at least below the knee, and a heel cup having side walls, a rear wall, and a bottom wall defining a recess located in the heel area, said heel cup being shaped to fit about the sides and rear of a wearer's heel, wherein said heel cup is secured to the lower leg and ankle brace at least about the peripheral edge of the heel cup to form an integral assembly therewith to secure the heel cup to the heel portion of the wearer's foot whereby the leg brace supports the wearer's lower leg and ankle and said heel cup is maintained in a controlled position on the foot to absorb shock.

### BRIEF DESCRIPTION OF THE DRAWINGS

FIG. 1 is a perspective view of an embodiment of the claimed subject matter as worn on the leg, ankle and foot of a wearer;

FIG. 2 is a plan view of the interior of an embodiment of the claimed subject matter in a flattened condition prior to assembly;

FIG. 3 is a sectional view taken along lines 3-3 of FIG. 2;

FIG. 4 is an plan view of the exterior of an embodiment of the claimed subject matter in a flattened condition prior to assembly;

FIG. 5 is a detail view of the shock absorbing structure of FIG. 4;

FIG. 6 is an elevational view showing the device in FIG. 1 in section and in position in relationship to the bone structure of the lower leg, ankle and foot as it is applied to the lower leg, ankle and foot of the wearer.

### DETAILED DESCRIPTION OF THE EMBODIMENTS

The skeletal structure of the human foot and ankle includes the lower portion of the tibia and fibula and the os calcis or heel bone and the talus is positioned above the os calcis and below the tibia. The forward bone structure of the foot includes the cuboid and metatarsal bones extending forwardly from the cuboid.

Body weight is normally supported at three points in the foot: the two weight-bearing portions on the forward part of the foot in the metatarsal area and the bottom of the foot below the os calcis. Running, jumping and walking often impose heel strike forces of substantial magnitude on the os calcis which are transmitted to the leg structure of the individual. These heel strike forces translate into force that wedges the talus bone between the tibia and fibula bones resulting in the stretching and subsequent inflammatory reaction of the deep tissues of the lower leg including the interossius membrane. ‘Shin splints,’ are the symptoms of pain felt over the anterior tibial crest of the lower legs. The inflammatory reaction occurs at the point where the deep tissues insert into the medial or anterior aspect of the tibia bones, and pain is typically felt between 3 and 16 cm above the foot.

Embodiments of the claimed subject matter address the pain of shin splints by maintaining the relative positions of the tibia and fibula bones so that they do not separate and irritate the surrounding lower leg tissues.

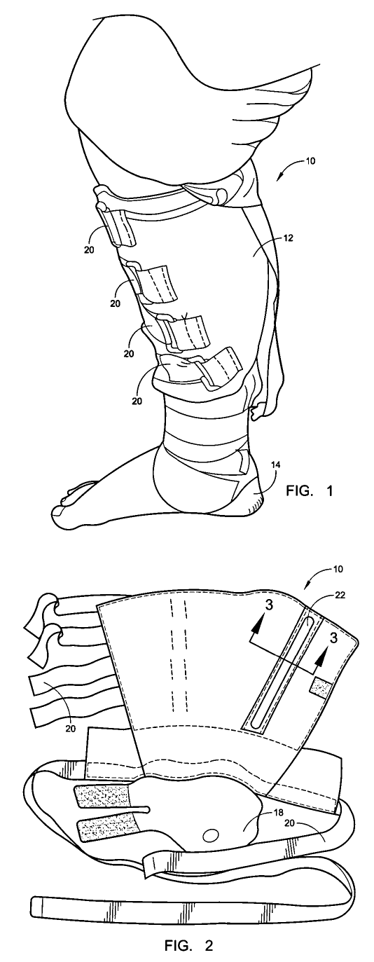

One embodiment illustrated in FIGS. 1 through 6, generally designated by the numeral 10, is a combined heel cup and lower leg brace that prevents the separation of the tibia and fibula when force is applied to the heel.

Article 10 is designed to fit snugly about the lower leg and heel areas of the wearer and it includes a lower leg and ankle brace 12 and a heel cup 14. Lower leg and ankle brace 12 receives the foot, ankle and lower leg of the wearer. It is generally tubular in shape extending downwardly from below the knee conforming to the general shape of the lower leg, ankle and heel of the user. FIG. 1. shows the upper edge of the brace 12 extending from an area from just below the knee to the heel of the wearer. Other embodiments may extend from an area further below the knee to the heel.

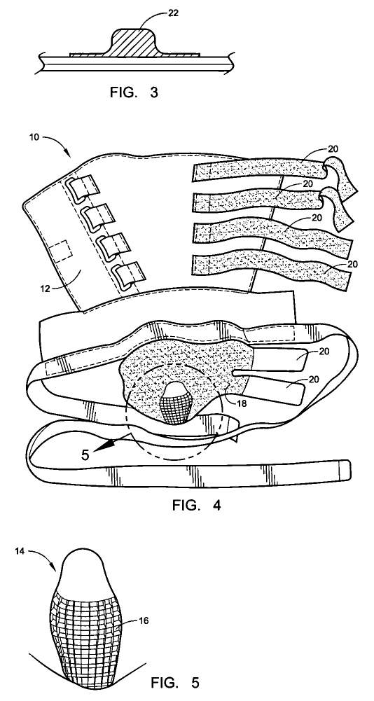

The leg brace 12 may be constructed from a stretchable material such as a natural latex rubber material, spandex or an expanded neoprene of the type commonly used in such applications as diver's wetsuits. It may also be constructed of any other suitable material so that the material has elastic properties sufficient to allow the material to stretch around the lower leg, ankle and heel in order to provide suitable support. The brace 12 may also extend further around the non-heel areas of the wearer's foot for additional support. The material may also be a laminate material with suitable laminations to allow the material to properly breath, absorb moisture and resist wear. In other embodiments, a stretchable sock or stocking may be used. Additionally, as illustrated in FIGS. 2 and 3, support section 22 may be used for additional support in brace 12.

The heel cup 14 may be constructed from a shock absorbing material, such as molded rubber, formed into a heel-like body. Any commercially available heel cup may be used so long as it can be secured to the leg and ankle brace 12 which acts as a tibial-fibular stabilizer.

In the illustrated embodiment, FIGS. 4 and 5 show the heel cup 14 as being integrally molded into ankle portion 18 of leg and ankle brace 12. The heel cup 14 has a heel-receiving interior recess and a vertical rear wall that is integral with the vertical rear wall of leg and ankle brace 12. In this embodiment, shock absorbing transverse ribs 16 are provided on the exterior surface of the cup-like body. The exterior of the cup-like body may be constructed with other shock absorbing materials or means or with no shock absorbing materials or means. The illustrated transverse ribs 16 intersect the longitudinal ribs at spaced intervals forming a plurality of generally rectangular shock absorbing sections having a waffle-like appearance. Shock forces imparted during walking, running or other activities are absorbed by deflection or deformation of the ribs. After deflection, the ribs return to their normal, nondeflected state.

The heel cup 14 may be integral to the leg and ankle brace 12 or it may be separate from the leg and ankle brace 12. If separate, it may be removably secured to the leg and ankle brace 12. The heel cup 14 may be constructed of a suitable light-weight material such as natural latex rubber, neoprene or low-density thermoplastic material such as polypropylene or polyethylene. The particular materials and fabrication may vary but the particular material should be resilient and have memory so that after the material has deformed it returns to its original shape or condition.

In some embodiments, the shock absorbing heel cup 14 is attached to the lower leg and ankle brace 12 by an appropriate adhesive or it is sewn into leg brace 12. Referring to FIGS. 3 and 4, FIG. 2 is a plan view of the interior of an embodiment of article 10 in a flattened condition prior to assembly. Lines 3-3 illustrate the location of the cross section of the reinforced support material 22 which is constructed of a suitable material or fabric which supports the leg and ankle brace 12. FIG. 3 is a sectional view taken along lines 3-3 of FIG. 2 showing the cross section of reinforced support 22.

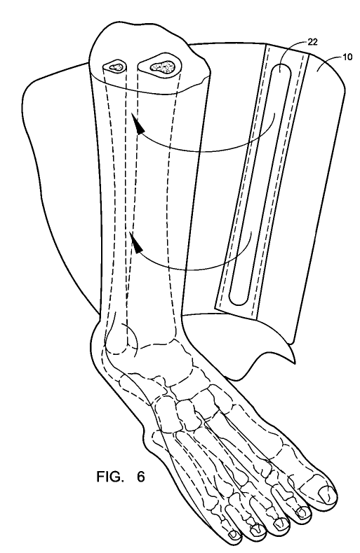

In use, the lower leg of the wearer's body is inserted in the upper end of the leg and ankle brace 12 of the article 10. FIG. 6 illustrates an embodiment of the claimed subject matter as it would be wrapped around the wearer's lower leg. The upper edge of the article 10 is grasped and pulled so that the article 10 assumes a position about the wearer's lower leg and ankle as well as the wearer's heel. As the straps 20 are tightened, the wearer's heel is firmly seated in the heel cup 14 of article 10.

A portion of the wearer's foot projects forwardly from the front edge of the leg and ankle brace 12 that encompasses the wearer's foot. The elasticized lower leg and ankle brace 12 provides support to the wearer and may also help to minimize swelling in the areas of the lower leg and ankle. The leg and ankle brace 12 is again constructed so it has elastic characteristics to enable it to stretch and snugly engage the lower leg, ankle and heel of the wearer. In other embodiments, a plurality of ventilation openings may be provided throughout article 10 at suitable positions for the comfort of the wearer.

Force is transmitted after impact from the heel cup 14 to the lower leg and ankle brace 12 lessening the spreading of the tibia and fibula bones resulting in less or no irritation of the surrounding lower leg tissues.

As previously described, the illustrated embodiment of the lower leg and ankle brace 12 is fabricated having upper edge which extends to an area at or below the knee of the wearer. Embodiments may also be constricted in various lengths that are suited for various individual wearers. Other embodiments may use a shorter length leg and ankle brace 12 wherein the brace 12 does not reach all the way to the base of the knee. A high top embodiment may be used to add support material to the wearer's ankle. Similarly, a low top embodiment may be used for minimal ankle support while still maintaining the shock absorbing properties of the present embodiments.

The ankle support portion 18 of the leg and ankle brace 12, as shown in FIGS. 2 and 4, may include one or more straps 20 for securing the ankle portion to the wearer's ankle. Additional opening and closing fasteners may be provided to allow the ankle support portion 18 to permit the portion 18 to be separately opened and closed so that it may be adjusted into a snug position. Any suitable type of closures such as snaps or loop and nook fabric fasteners may be used for this purpose. The position of the closure may also be provided at other locations such as at the medial or lateral sides of the portion 18. Other straps may be stitched or otherwise secured to the leg brace 12 about the lower leg and/or about the ankle to provide additional support to the wearer.

In the embodiment shown in FIG. 1, the wearer may position and tighten the straps around the lower leg to provide additional support as desired. The distal ends of the straps extend at least partially about the lower leg and are securable at fastener sections on the straps 20 at one or more predetermined locations on the lower leg and ankle brace 12. Lower leg and ankle brace 12 may also be constructed of a single sleeve with no straps, with a single strap 20, or with more than one straps 20.

Thus, it will be seen that embodiments of the claimed subject matter provide a simple, effective and unique support and cushioning article for the foot and leg of the wearer that helps prevent or eliminate the painful effects of shin splints.

Any form or configuration of the leg and ankle brace 12 and heel cup 14 may be used with embodiments of the present invention. For example, the heel cup 14 may not be secured to the brace 12 as described, but rather enveloped in a portion of brace 12 leading to the same effect of force being transferred to the brace 12. The described configurations were selected for illustrative purposes. Any suitable material may also be used in the construction of the lower leg and ankle brace 12 and the heel cup 14 attached thereto.

Although the invention has been described herein by reference to exemplary embodiments thereof, it will be understood that such embodiments are susceptible of modification and variation without departing from the inventive concepts disclosed. All such modifications and variations, therefore, are intended to be encompassed within the spirit and scope of the appended claims.

1. An article for treatment and prevention of shin splints comprising a lower leg and ankle brace having a generally tubular body with an upper and a lower end shaped to fit about a wearer's leg extending from the bottom of the foot to at least below the knee; and a heel cup having side walls, a rear wall, and a bottom wall defining a recess located in the heel area, said heel cup being shaped to fit about the sides and rear of a wearer's heel; wherein said heel cup is secured to said lower leg brace at least about the peripheral edge of the heel cup to form an integral assembly therewith to secure the heel cup to the heel portion of the wearer's foot whereby said leg brace supports the wearer's lower leg and ankle and said heel cup is maintained in a controlled position on the foot to absorb shock.

2. The article of claim 1 wherein said lower leg brace further comprises one or more straps to secure the wearer's ankle in position.

3. The article of claim 1 wherein said lower leg brace further comprises one or more straps to secure the wearer's lower leg in position.

4. The article of claim 1 wherein said heel cup extends a distance along the foot substantially conforming to the plantar surface of the foot and terminating in the metatarsal area of the foot.

5. The article of claim 1 wherein said lower leg and ankle brace is constructed of neoprene.

6. The article of claim 1 wherein said heel cup is constructed of molded rubber.

7. The article of claim 1 wherein said lower leg and ankle brace is provided with vent openings.

8. The article of claim 1 wherein said lower leg and ankle brace is provided with one or more fastenable closures.

9. The article of claim 8 wherein said one or more closures are fabric hook and loop fastener members.

10. The article of claim 1 wherein said lower leg and ankle brace is secured to said heel cup with one or more straps.

11. The article of claim 1 wherein said heel cup is formed as an integral part of footwear.

12. The article of claim 1 wherein said heel cup is formed as an integral part of footwear insole.

13. The article of claim 1 wherein said heel cup is constructed of a shock absorbing material.

14. The article of claim 1 wherein said heel cup is removably secured to the lower leg and ankle brace.
